(19) United States
(12) Patent Application Publication
(10) Pub. No.: US 2024/0133572 A1
(43) Pub. Date: Apr. 25, 2024

(54) SYSTEM FOR DIAGNOSIS AND MANAGEMENT OF INDOOR AIR QUALITY USING MACHINE LEARNING

(71) Applicant: (RT DATA LAB CO., LTD.), Seoul (KR)

(72) Inventors: Yoo Sin KIM, Seoul (KR); Eun Joo PARK, Yongin-si (KR); Jung Hoon SHIN, Yongin-si (KR); Tae Yun KIM, Cheongju-si (KR); Kun Wook KOH, Seoul (KR)

(73) Assignee: AirDeep Co., Ltd., Seoul (KR)

(21) Appl. No.: 18/277,510

(22) PCT Filed: Nov. 25, 2021
(86) PCT No.: PCT/KR2021/017555

§ 371 Date: Aug. 15, 2023

(30) Foreign Application Priority Data
Feb. 24, 2021 (KR) 10-2021-0024693
Aug. 9, 2021 (KR) 10-2021-0105311

(51) Int. Cl. F24F 11/64; G06N 20/00

(52) U.S. Cl. CPC F24F 11/64; G06N 20/00; F24F 2110/62

(57) ABSTRACT

The present invention relates to a system for diagnosis and management of indoor air quality, and more particularly to a system for diagnosis and management of indoor air quality which is capable of obtaining and analyzing air quality data by measuring the indoor air quality of a vehicle or accommodation space and of detecting smoking or non-smoking, the type of smoking, and an abnormal situation by diagnosing at least one of indoor smoking and smoking types.

In addition, the present invention relates to a technology for facilitating the management of accommodation by efficiently diagnosing indoor air quality.

### TECHNICAL FIELD

This application claims priority to Korean Patent Application No. 10-2021-0024693, filed on Feb. 24, 2021, and Korean Patent Application No. 10-2021-0105311, filed on Feb. 24, 2021, in the Korean Intellectual Property Office, the disclosures of each of which are incorporated herein by reference.

The present invention relates to a system for diagnosis and management of indoor air quality, and more particularly to a system for diagnosis and management of indoor air quality which is capable of obtaining and analyzing air quality data by measuring the indoor air quality of a vehicle or accommodation space and of detecting smoking or non-smoking, the type of smoking, and an abnormal situation by diagnosing at least one of indoor smoking and smoking types.

In addition, the present invention relates to a technology capable of facilitating the management of accommodation facilities by efficiently diagnosing indoor air quality.

### BACKGROUND ART

Recently, smoking cessation is recommended due to health problems such as lung cancer caused by smoking. In particular, smoking in a limited indoor space such as a vehicle or a room causes great pain to non-smokers due to secondhand smoke and adversely affects air quality.

Accordingly, despite strict prohibition of smoking indoors such as a vehicle or a room, some smokers still smoke indoors avoiding the eyes of others.

Cigarette smoke spreads in a fairly wide range along the flow of air, and even if someone smokes a cigarette indoors, it is difficult to detect it immediately. When others notice smoking and search for the smoker, it is usually after the smoker has finished smoking and disappeared. That is, if smoking is detected using manpower, it is difficult to quickly grasp it, and there is a limit to effectiveness.

Therefore, there is a need for a system capable of immediately detecting and notifying smoking when a smoker smokes a cigarette indoors.

On the other hand, in the case of conventional smoking detection systems, it is possible to detect only tobacco cigarettes before the appearance of electronic cigarettes, and in the case of electronic cigarettes (including both cigarette and liquid types), smoke components are different, so they cannot be detected in many cases. Accordingly, demand for a system capable of detecting both tobacco cigarettes and electronic cigarettes is also increasing.

Furthermore, in room management of an accommodation facility, it is difficult for a user to quickly intervene and perform management measures even if smoking occurs indoors during a period of use by a customer. Accordingly, there is a problem in that a belated discovery and action are taken for a situation in which room management needs to be performed, such as deterioration of room conditions or occurrence of an abnormal situation.

In addition, in the case of large accommodations, room management and room control systems linked to the use history of numerous customers visiting the large accommodations have not yet been applied, making it impossible to systematically manage rooms for each customer's characteristics. In addition, there is a problem that it is difficult for a manager to promptly judge and respond to each situation when situations requiring action occur frequently.

### DISCLOSURE

### Technical Problem

Therefore, the present invention has been made in view of the above problems, and it is one object of the present invention to provide a system for diagnosis and management of indoor air quality which is capable of obtaining and analyzing air quality data by measuring the indoor air quality of a vehicle or accommodation space and of detecting smoking or non-smoking, the type of smoking, and an abnormal situation by diagnosing at least one of indoor smoking and smoking types.

It is another object of the present invention to provide a system for diagnosis and management of indoor air quality capable of classifying the types of customers who use accommodations by analyzing the management data built for accommodations and the air quality data of accommodations, capable of providing analysis information according to each customer type to the manager, and enabling real-time control of accommodation room status and management according to customer's accommodation usage history.

### Technical Solution

In accordance with an aspect of the present invention, the above and other objects can be accomplished by the provision of a system for diagnosis and management of indoor air quality, including: an air quality measurer configured to obtain air quality data by measuring indoor air quality in a limited space; an air quality analyzer configured to analyze the obtained air quality data based on a machine learning result of the air quality data according to smoking or not; and an indoor air diagnotor configured to diagnose one or more of indoor smoking or not and smoking types based on the air quality data analysis result.

Preferably, the air quality measurer may be an air quality sensor (AQS).

Preferably, the air quality analyzer may include a detection model generator that creates a smoking detection model capable of detecting indoor smoking by learning smoking data, which is air quality data when smoking is performed indoors, and non-smoking data, which is air quality data when smoking is not performed indoors and that creates a smoking type classification capable of classifying smoking types by learning tobacco data, which is air quality data when smoking is performed with a tobacco cigarette indoors, and cigarette data which is air quality data when smoking is performed with an electronic cigarette model.

Preferably, the detection model generator may create the smoking detection model or the smoking type classification model using one or more models selected from supervised learning models including decision tree, random forest, Extreme Gradient Boosting (XGBOOST) and Support Vector Machine (SVM).

Preferably, the detection model generator may select an optimal parameter in one or more manners of grid search and cross validation in parameters (hyperparameter) applied to the supervised learning models.

Preferably, the indoor air diagnotor may determine that there is a smoker indoors when smoking is detected a first set number of times within a first set time by the smoking detection model, and may classify a smoking type by the smoking type classification model, sum the number of times classified as a tobacco cigarette and the number of times classified as an electronic cigarette, and diagnose a smoking type based on the number of times more than half of the number of times classified as a tobacco cigarette and the number of times classified as an electronic cigarette.

Preferably, when determining that there is a smoker indoors, the indoor air diagnotor may not detect smoking for a second set time from a first smoking detection point after the first set number of times of smoking detection.

Preferably, the indoor air diagnotor may determine that air quality is poor when an ECO2 value measured by the air quality measurer is a first reference value and a TVOC value measured thereby is a second reference value or more or when a PM10 value is a third reference value or more and a PM2.5 value is a fourth reference value or more.

Preferably, the first reference value may be 1000 μg/m³, the second reference value may be 1300 μg/m³, the third reference value may be 80 μg/m³, and the fourth reference value may be 35 μg/m³.

Preferably, the indoor air diagnotor may determine as an abnormal situation when an ECO2 value measured by the air quality measurer is confirmed as a fifth reference value or more, a TVOC value is confirmed as a sixth reference value or more, a PM10 value is confirmed as a seventh reference value or more, and a PM2.5 value is confirmed as an eighth reference value or more a second set number of times within a third set time.

Preferably, the fifth reference value may be 3500 μg/m³, the sixth reference value may be 4000 μg/m³, the seventh reference value may be 1700 μg/m³, and the eighth reference value may be 1700 μg/m³.

Preferably, the system for diagnosis and management of indoor air quality may further include a notification signal output configured to generate and output a notification signal according to an air diagnosis result of the indoor air diagnotor.

In accordance with another aspect of the present invention, there is provided a method of diagnosing indoor air quality, the method including: an air quality measurement step of obtaining air quality data by measuring indoor air quality in a limited space; an air quality analyzing step of analyzing the obtained air quality data based on a machine learning result of the air quality data according to smoking or not; and an indoor air diagnosis step of diagnosing one or more of indoor smoking or not and smoking types based on the air quality data analysis result.

Preferably, the air quality analysis step may include a detection model generation step of creating a smoking detection model capable of detecting indoor smoking by learning smoking data, which is air quality data when smoking is performed indoors, and non-smoking data, which is air quality data when smoking is not performed indoors and that creates a smoking type classification capable of classifying smoking types by learning tobacco data, which is air quality data when smoking is performed with a tobacco cigarette indoors, and cigarette data which is air quality data when smoking is performed with an electronic cigarette model.

Preferably, the detection model generation step may include a step of creating the smoking detection model or the smoking type classification model using one or more models selected from supervised learning models including decision tree, random forest, Extreme Gradient Boosting (XGBOOST) and Support Vector Machine (SVM).

Preferably, the detection model generation step may further include a step of selecting an optimal parameter in one or more manners of grid search and cross validation in parameters (hyperparameter) applied to the supervised learning models.

Preferably, the indoor air diagnosis step may include a step of determining that there is a smoker indoors when smoking is detected a first set number of times within a first set time by the smoking detection model, and a step of classifying a smoking type by the smoking type classification model, summing the number of times classified as a tobacco cigarette and the number of times classified as an electronic cigarette, and diagnosing a smoking type based on the number of times more than half of the number of times classified as a tobacco cigarette and the number of times classified as an electronic cigarette.

Preferably, when determining that there is a smoker indoors, the indoor air diagnosis step may further include a step of not detecting smoking for a second set time from a first smoking detection point after the first set number of times of smoking detection.

Preferably, the indoor air diagnosis step may include a step of determining that air quality is poor when an ECO2 value measured by the air quality measurement step is a first reference value and a TVOC value measured thereby is a second reference value or more or when a PM10 value is a third reference value or more and a PM2.5 value is a fourth reference value or more.

Preferably, the first reference value may be 1000 μg/m³, the second reference value may be 1300 μg/m³, the third reference value may be 80 μg/m³, and the fourth reference value may be 35 μg/m³.

Preferably, the indoor air diagnosis step may include a step of determining as an abnormal situation when an ECO2 value measured by the air quality measurement step is confirmed as a fifth reference value or more, a TVOC value is confirmed as a sixth reference value or more, a PM10 value is confirmed as a seventh reference value or more, and a PM2.5 value is confirmed as an eighth reference value or more a second set number of times within a third set time.

Preferably, the fifth reference value may be 3500 μg/m³, the sixth reference value may be 4000 μg/m³, the seventh reference value may be 1700 μg/m³, and the eighth reference value may be 1700 μg/m³.

Preferably, the method of diagnosing indoor air quality may further include a notification signal output step of generating and outputting a notification signal according to an air diagnosis result of the indoor air diagnosis step.

In accordance with yet another aspect of the present invention, there is provided a system for diagnosis and management of indoor air quality, including: an air quality measurer configured to obtain air quality data for each room in accommodation; an air quality analyzer configured to create a smoking detection model and a smoking type classification model and analyze the obtained air quality data based on the machine learning results of the air quality data; a management data constructor configured to construct management data for the accommodation; and a customer type classifier configured to classify types of customers using the accommodation based on the analysis results of the management data and the air quality data, wherein the customer type classifier classifies the customer into any one of a smoking customer group, a non-smoking customer group and other customer groups, subclasses a customer corresponding to the smoking customer group into a first smoking customer or a second smoking customer, and subclasses customers belonging to each customer group through logistic regression or cluster analysis.

Preferably, the management data constructor may construct the management data based on one or more of an accommodation use history of the customer and a smoking history of the customer.

Preferably, the customer type classifier may subclass customers corresponding to the non-smoking customer group into a normal customer, a cautionary customer, or a risky customer.

Preferably, the customer type classifier may perform the logistic regression or cluster analysis by applying independent variables extracted from the management data.

Preferably, the customer type classifier may set a criteria for subclassing the customers as a dependent variable and perform the logistic regression or cluster analysis.

Preferably, the customer type classifier may perform the logistic regression or the cluster analysis to subclass a customer, who corresponds to an interval with a standard deviation twice or more than an average, among customers belonging to the smoking customer group into the second smoking customer, and may perform the logistic regression or the cluster analysis to subclass a customer, who falls within an interval excluding intervals that are twice the standard deviation or more than an average, among customers belonging to the smoking customer group into the first smoking customer.

Preferably, the customer type classifier may perform the logistic regression or the cluster analysis to subclass a customer, who falls into the range of 3 times a standard deviation or more than an average, among customers belonging to the non-smoking group, into the risky customer, and may perform the logistic regression or the cluster analysis to subclass a customer, who falls within an interval excluding an interval that is 1 times the standard deviation or more than an average, among customers belonging to the non-smoking customer group into the normal customer.

Preferably, the system may further include a control alarm transmitter configured to deliver a control alarm for each room of the accommodation to the customer based on the type of customer to be classified; and an analysis information provider configured to provide reporting-type analysis information to the accommodation manager based on the classified customer type.

### Advantageous Effects

In accordance with an aspect of the present invention, air quality data can be obtained by measuring the indoor air quality in a limited space, the obtained air quality data can be analyzed based on a machine learning result of the air quality data according to smoking status, and not only indoor smoking status but also types of smoking such as tobacco cigarettes and electronic cigarettes can be determined through measurement of indoor air quality by diagnosing at least one of indoor smoking status and smoking type according to the air quality data analysis result, it is possible to determine. Furthermore, abnormal situations such as fire can be detected.

In accordance with another aspect of the present invention, information on customers included in a customer group related to the history of abnormalities in each room of accommodations and the corresponding rooms is provided to a manager through a control service, so the manager can easily check the history of abnormalities in each room to manage customers and rooms.

In accordance with yet another aspect of the present invention, when abnormal behavior such as smoking in a room is detected, a control alarm is delivered to a customer in real-time, which has the effect of preventing the illegal use of accommodation.

### DESCRIPTION OF DRAWINGS

FIG. 1 schematically illustrates a system for diagnosis and management of indoor air quality according to an embodiment of the present invention.

FIG. 2 explains Support Vector Machine (SVM) among supervised learning models used in a system for diagnosis and management of indoor air quality according to an embodiment of the present invention.

FIG. 3 is a diagram for explaining cross validation among methods for selecting an optimal parameter applied to a supervised learning model used in a system for diagnosis and management of indoor air quality according to an embodiment of the present invention.

FIG. 4 is a diagram for explaining a confusion matrix among evaluation indexes for evaluating a supervised learning model and parameter used in a system for diagnosis and management of indoor air quality according to an embodiment of the present invention.

FIG. 5 is a drawing for explaining an embodiment of a process of diagnosing indoor air quality by an indoor air diagnotor of a system for diagnosis and management of indoor air quality according to an embodiment of the present invention.

FIG. 6 is a flowchart for explaining a method of diagnosing indoor air quality according to an embodiment of the present invention.

FIG. 7 is a reference diagram for explaining a system for diagnosis and management of indoor air quality according to another embodiment of the present invention.

FIG. 8 is a block diagram illustrating the composition of a service-providing device 10 according to the present invention.

FIGS. 9 and 10 are reference diagrams for explaining a process of classifying customer types using accommodation according to another embodiment of the present invention.

FIG. 11 is a reference diagram illustrating a control alarm delivered to a customer using accommodation according to another embodiment of the present invention.

### BEST MODE

The present invention will be described in detail with reference to the accompanying drawings. Here, detailed descriptions of repetitive descriptions and known functions and configurations that may unnecessarily obscure the gist of the present invention are omitted. Embodiments of the present invention are provided to more completely explain the present invention to those skilled in the art. Accordingly, the shapes and sizes of elements in the drawings may be exaggerated for clarity.

Throughout the specification, when a certain component is said to “comprise” a certain component, it means that it may further comprise other components without excluding other components unless otherwise stated.

In addition, the term “part” described in the specification means a unit that processes one or more functions or operations, which may be implemented as hardware or software or a combination of hardware and software.

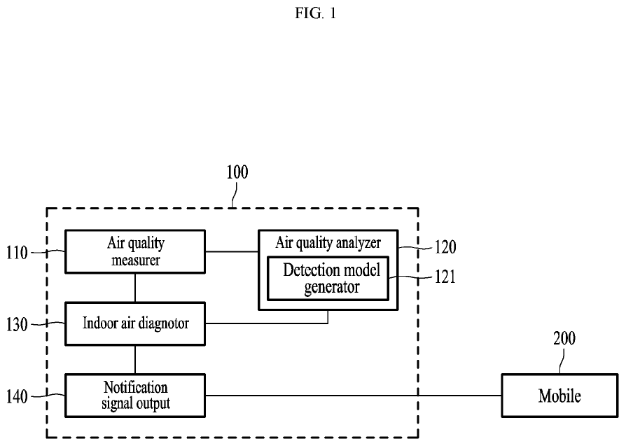

FIG. 1 schematically illustrates a system for diagnosis and management of indoor air quality according to an embodiment of the present invention.

Referring to FIG. 1, the system 100 for diagnosis and management of indoor air quality according to an embodiment of the present invention is communicatively connected to a user's mobile 200 to transmit and receive signals. The system 100 for diagnosis and management of indoor air quality according to an embodiment of the present invention includes an air quality measurer 110, an air quality analyzer 120, an indoor air diagnotor 130 and a notification signal output 140. The system 100 for diagnosis and management of indoor air quality shown in FIG. 1 is an embodiment, and the components shown in FIG. 1 are not intended to limit the embodiment shown in FIG. 1 and may be added, changed, or deleted as necessary.

The air quality measurer 110 may obtain air quality data by measuring indoor air quality in a limited space. In an embodiment, In one embodiment, the limited space means a space partitioned by walls, doors, etc., such as the inside of a vehicle or a room. The air quality measurer 110 may be an air quality sensor (AQS). An air quality sensor is a sensor that measures the levels of harmful substances such as ECO2, TVOC, PM10, and PM2.5 in the air. Air quality data generated by measuring indoor air quality by the air quality measurer 110 is used for machine learning in the air quality analyzer 130 or used to analyze air quality data.

The air quality analyzer 120 may analyze the obtained air quality data based on a machine learning result of the air quality data according to smoking or not. In an embodiment, the air quality analyzer 120 may include a detection model generator 121 that creates a smoking detection model capable of detecting indoor smoking by learning smoking data, which is air quality data when smoking is performed indoors, and non-smoking data, which is air quality data when smoking is not performed indoors or that creates a smoking type classification capable of classifying smoking types by learning tobacco data, which is air quality data when smoking is performed with a tobacco cigarette indoors, and cigarette data which is air quality data when smoking is performed with an electronic cigarette model.

The detection model generator 121 may create one or more of a smoking detection model that can detect indoor smoking by analyzing air quality data measured by the air quality measurer 110 and performing machine learning; and a smoking type classification model that can classify a smoking type. Here, the detection model generator 121 may create the smoking detection model or the smoking type classification model using one or more models selected from supervised learning models including decision tree, random forest, Extreme Gradient Boosting (XGBOOST) and Support Vector Machine (SVM). A model by the detection model generator 121 is described below.

Decision Tree

A decision tree is a supervised learning model that classifies or predicts the entire data into small groups according to conditions, and its appearance is a tree branch structure. The decision tree has the advantage of being easy to explain conditions and branching, as it is possible to see through visualization what specific criteria the data were divided into.

Regarding the tree branch structure, the case of data that is not divided at all is called a root node, a group divided according to conditions is called a child node, and a group that is not further divided is called a final node.

If the form of a class (an answer to be obtained through the model) to be finally derived is discrete (ex. 0, 1, 2, . . . ), it is called a classification tree; otherwise, if it is continuous (ex. continuous number type such as 10.23 and 14.56), it is called a regression tree.

the detection model generator 121 of the indoor air diagnosis system 100 according to an embodiment of the present invention uses a decision tree among supervised learning models, a classification tree is used because the purpose is to classify whether one case of air quality data is smoking or not, and whether it is an electronic cigarette or a tobacco cigarette.

In the case of the classification tree, one of indicators such as the p-value of the chi-square statistic of the values of variables used, the Gini index, and the entropy index is used when continuously dividing data.

As a p-value is small, the heterogeneity between the divided nodes increases, and as the values of the Gini index and the entropy index are small, the heterogeneity between data within a node decreases. The model sets conditions in a direction in which heterogeneity between nodes is high and heterogeneity between data within a node is small.

Since there are various options for the criterion for dividing nodes, a researcher can choose according to a direction they want.

However, the decision tree has a disadvantage in that it is difficult to generalize and use results because a deviation between models is large depending on learning data, and the random forest technique overcomes this disadvantage.

Random Forest

A random forest is a supervised learning model based on a decision tree, and follows a method (bagging among ensemble techniques) that selects the most frequently appearing result among results obtained through multiple decision trees.

To overcome the data dependence of existing decision trees, the random forest selects N data through restoration extraction for the training data, and learns by randomly selecting d variables (e.g., measurement values of an air quality sensor) considered for prediction or classification. In this way, prediction and classification are performed through K decision trees. An average of each K results or the most frequently appearing result is determined as a final value.

In an embodiment, when creating a random forest and other various machine learning models using the Python 3 language, prediction and classification results may vary depending on several hyperparameters (model settings). A model is optimized by selecting a hyperparameter that gives the best results through multiple learning (experiments).

The detection model generator 121 of the indoor air diagnosis system 100 according to an embodiment of the present invention can examine hyperparameters such as the number of decision trees, the maximum number of variables that can be extracted, the depth of the decision tree, the minimum number of data constituting one node, and the minimum number of data required to constitute a final node, and can be selected through a grid search technique. The following table shows an example of a hyperparameter of a supervised learning model that can be used in the detection model generator 121 of the indoor air diagnosis system 100 according to an embodiment of the present invention.

TABLE 1

| Parameter name | Description |
|---|---|
| n_estimator | • The number of decision trees, default is 10 |
| | • The performance may be better as the number is |
| | large, but it is not unconditional. |
| max_features | • Default = 1 • Set to 1 if it is not desired |
| | to display output messages. |
| max_depth | • Adjust the number of CPU execution threads • All |
| | defaults are used. |
| | • Changed when using only some CPUs in a multi- |
| | core/thread CPU system. |
| min_sample_leaf | § Minimum number of sample data to become a |
| | leaf node |
| min_samples_split | § Minimum number of data to split a node |

XGBOOST

Extreme Gradient Boosting (XGBOOST) is a supervised learning model that has the advantage of being relatively fast by applying a gradient boosting technique based on a decision tree and implementing it to run in a distributed environment.

Here, boosting, which is one of ensemble techniques, is a method of improving errors by assigning weights to incorrectly predicted data while sequentially learning and predicting/classifying several models. Gradient boosting uses a gradient descent method when updating weights. The gradient descent method initializes a value by subtracting the derivative of a cost function from an initial value, and repeats until the derivative is minimized.

Since XGBOOST is highly likely to have different performance depending on learning parameters, it can be optimized while experimenting with various parameters.

SVM

Support Vector Machine (SVM) is a supervised learning model that defines a baseline for classification of data. Depending on the number of attributes, the method of drawing a baseline for classification also changes. In this case, the baseline is also called a decision boundary.

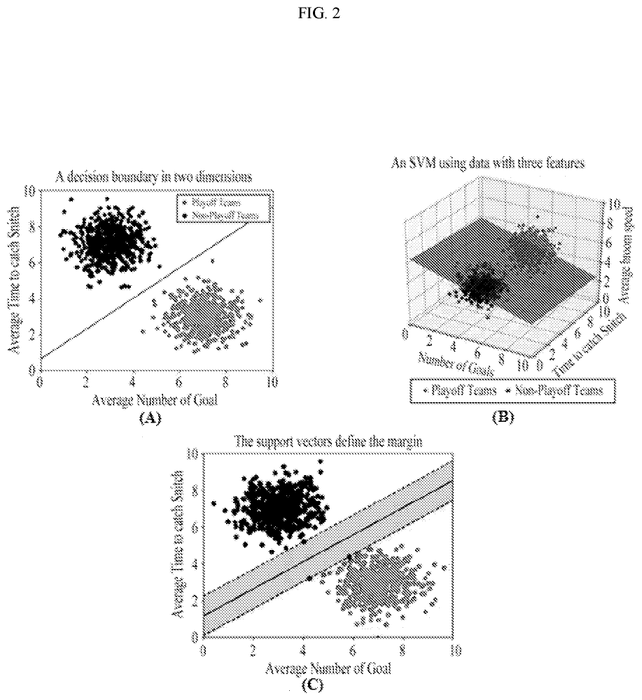

FIG. 2 explains Support Vector Machine (SVM) among supervised learning models used in a system for diagnosis and management of indoor air quality according to an embodiment of the present invention.

Referring to FIG. 2, classification may be performed in a two-dimensional graph for example if there are two values of variables used for classification as in (A) of FIG. 2 (ex. if only PM2.5 and PM10 are used). In addition, if the value of a variable increases to three (ex. PM2.5, PM10, TVOC) as in (B) of FIG. 2, it should be classified in a three-dimensional graph. Here, a decision boundary becomes a plane rather than a line. In the case of a supervised learning model that can be used in the detection model generator 121 of the indoor air diagnosis system 100 according to an embodiment of the present invention, it can learn using three features, and if visualizing, it would have been classified as a picture like (B) of FIG. 2.

As shown in FIG. 2, the model also becomes more complex as the number of attributes increases, and a classification criterion “line (decision boundary)” also gradually changes to a higher dimension (this is called a hyperplane).

SVM basically learns in a direction of maximizing a margin. The margin means a distance between divided categories as shown in (C) of FIG. 2. As in (C) of FIG. 2, as the distance between the two categories is large, it represents better separation. A supervised learning model that can be used in the detection model generator 121 of the indoor air diagnosis system 100 according to an embodiment of the present invention may also perform learning for the purpose of maximizing the margin. As a result, the model of the embodiment showed the best accuracy in SVM.

Referring to FIG. 1 again, the detection model generator 121 may select an optimal parameter for a hyperparameter applied to a supervised learning model using one or more methods of grid search and cross validation. The method used to select a parameter in the detection model generator 121 is explained below.

Grid Search

Grid search is one of methodologies for exploring hyperparameters of an optimal model. It can search quickly and has the advantage of high efficiency. Grid search is a methodology that searches for an optimal parameter that can give the best score (based on evaluation index) by applying all possible combinations of hyperparameter candidates randomly designated by a user.

Cross Validation

Cross validation is an optimization methodology to prevent overfitting when available data set is small.

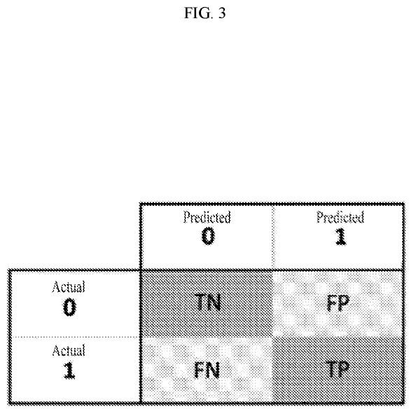

FIG. 3 is a diagram for explaining cross validation among methods for selecting an optimal parameter applied to a supervised learning model used in a system for diagnosis and management of indoor air quality according to an embodiment of the present invention.

As shown in FIG. 3, by dividing the entire data set into K constant values and evaluating K times, the data to be verified is not fixed and all data sets can be evaluated and verified, resulting in increased accuracy.

Referring to FIG. 1 again, the detection model generator 121 may evaluate accuracy using a confusion matrix to evaluate a supervised learning model and parameters used to generate a smoking detection model or a smoking type classification model. The confusion matrix is described below.

Confusion Matrix

A confusion matrix is a table used to check how well a classification problem is solved.

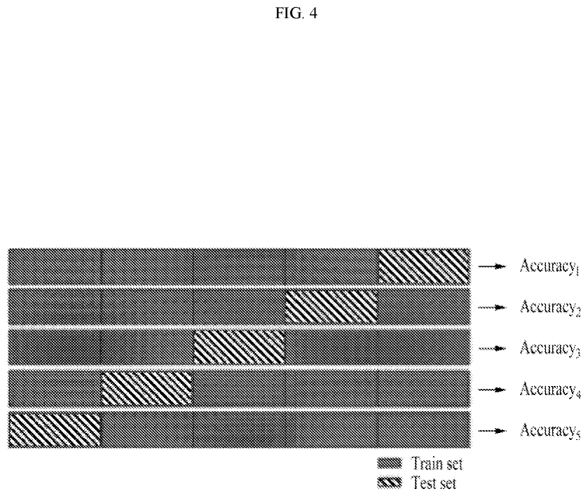

FIG. 4 is a diagram for explaining a confusion matrix among evaluation indexes for evaluating a supervised learning model and parameter used in a system for diagnosis and management of indoor air quality according to an embodiment of the present invention.

The column of FIG. 4 is a result predicted by the classification model, and the row is an actual value. If a predicted value is correct, it is displayed as TN, TP, and if it is incorrect, it is displayed as FP, FN.

Accuracy is an index that determines how similar predicted data is to actual data. In the case of supervised learning models that can be used in the detection model generator 121 of the indoor air diagnosis system 100 according to an embodiment of the present invention, accuracy may be used because, in practice, how many detections are more important than the pressure on false positives. A formula for accuracy is as follows:

$$Accuracy = \frac{TP + TN}{TP + FP + TN + FN} \qquad [ Equation 1 ]$$

As described above, the detection model generator 121 of the air quality analyzer 120 generates a smoking detection model, which can detect indoor smoking or not, or a smoking type classification model, which can classify smoking type, through machine learning, so that the indoor air diagnotor 130 can utilize the generated models to diagnose indoor air quality.

The indoor air diagnotor 130 diagnoses one or more of indoor smoking or not and a smoking type according to the air quality data analysis result of the air quality analyzer 120. In an embodiment, the indoor air diagnotor 130 may determine that there is a smoker indoors when smoking is detected a first set number of times within a first set time by the smoking detection model. For example, the indoor air diagnotor 130 can detect smoking by a smoking detection model. If smoking is detected three or more times within two minutes, it may be determined that there is a smoker indoors. Here, the first set time or the first set number of times may be set to an appropriate value to determine that there is a smoker indoors, and a user may set it as needed.

In an embodiment, the indoor air diagnotor 130 may classify a smoking type by the smoking type classification model, sum the number of times classified as a tobacco cigarette and the number of times classified as an electronic cigarette, and diagnose a smoking type based on the number of times more than half of the number of times classified as a tobacco cigarette and the number of times classified as an electronic cigarette. For example, the indoor air diagnotor 130 may diagnose a smoking type as a tobacco cigarette if the number of times classified as a tobacco cigarette is 3 and the number of times classified as an electronic cigarette is 1 within 2 minutes.

In an embodiment, when the indoor air diagnotor 130 determines that there is a smoker indoors, may not detect smoking for a second set time from the first smoking detection point after the first set number of times of smoking detection. For example, when smoking is detected three or more times within 2 minutes and it is determined that there is a smoker indoors, smoking may not be detected for 6 minutes from the time of the first smoking detection. The reason for not detecting smoking for a certain period of time is to prevent repeated detection of smoking for one smoking act. In one embodiment, a second set time is set to 6 minutes, which is a time set considering an average smoking time of 4 minutes and a normal air quality recovery time of 2 minutes. However, the second set time is not limited thereto and may be set to an appropriate value, and a user may set it as needed.

The indoor air diagnotor 130 may be determined that the air quality is poor when an ECO2 value measured by the air quality measurer 110 is a first reference value and a TVOC value measured thereby is a second reference value or more or when a PM10 value is a third reference value or more and a PM2.5 value is a fourth reference value or more. That is, when the measured value measured by the air quality measurer 110 exceeds a predetermined threshold value, the indoor air diagnotor 130 may determine that the air quality itself is bad rather than simply caused by smoking. For example, the first reference value may be 1000 μg/m³, the second reference value may be 1300 μg/m³, the third reference value may be 80 μg/m³, and the fourth reference value may be 35 μg/m³. 80 μg/m³ which is an example of the third reference value of PM10 and 35 μg/m³ which is an example of the fourth reference value of PM2.5 are values selected according to the air quality standards specified by the Korea Meteorological Administration. On the other hand, since the ratings of ECO2 and TVOC are not separately determined according to the Korean Meteorological Administration or international standards, an example of the first reference value of ECO2 and an example of the second reference value of TVOC were determined based on a value calculated by averaging the observed values of TVOC and ECO2 when PM10 and PM2.5 are at a poor air quality level in observed air quality values collected through experiments. The first to fourth reference values are not limited to the values given as examples, and the first to fourth reference values may be determined according to user's needs.

In addition, the indoor air diagnotor 130 may determine as an abnormal situation when an ECO2 value measured by the air quality measurer 110 is confirmed as a fifth reference value or more, a TVOC value is confirmed as a sixth reference value or more, a PM10 value is confirmed as a seventh reference value or more, and a PM2.5 value is confirmed as an eighth reference value or more the second set number of times within a third set time. Here, an abnormal situation may refer to a situation in which the concentration of harmful substances indoors becomes very high due to a fire or the like. For example, the fifth reference value may be 3500 μg/m³, the sixth reference value may be 4000 μg/m³, the seventh reference value may be 1700 μg/m³, and the eighth reference value may be 1700 μg/m³. The examples of the fifth to eighth reference values are examples of experimental measurement values when an abnormal situation is actually enough to trigger a carbon dioxide alarm. The fifth to eighth reference values are not limited to the values given as examples, and the fifth to eighth reference values may be set according to user's needs. Here, the fifth reference value is greater than the first reference value, the sixth reference value is greater than the second reference value, the seventh reference value is greater than the third reference value, and the eighth reference value is greater than the fourth reference value. Here, the third set time or the second set number of times may be set to an appropriate value to determine an abnormal situation, and may be set by a user as needed. For example, the third set time may be 2 minutes, and the second set number of times may be 3 times.

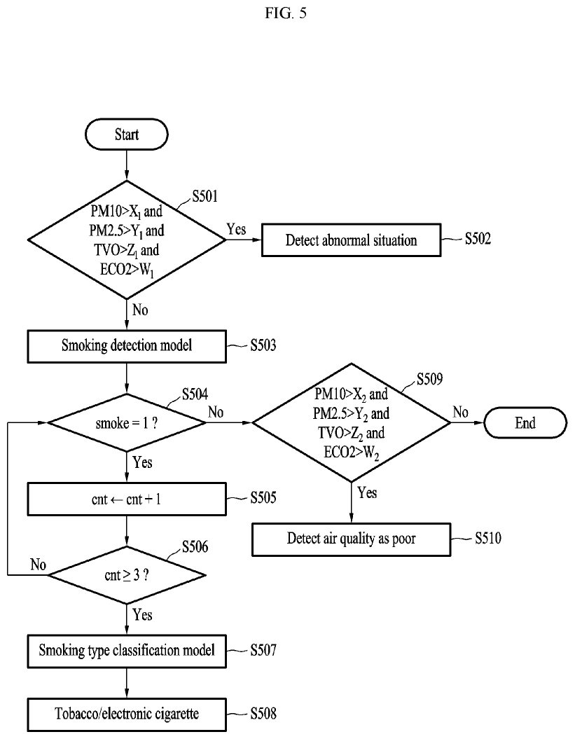

As described above, the process of diagnosing indoor air quality by the indoor air diagnotor 130 of the system 100 for diagnosis and management of indoor air quality according to an embodiment of the present invention has been described through an embodiment. Hereinafter, the process of diagnosing indoor air quality by the indoor air diagnotor 130 will be described in more detail with reference to FIG. 5.

FIG. 5 is a drawing for explaining an embodiment of a process of diagnosing indoor air quality by an indoor air diagnotor of a system for diagnosis and management of indoor air quality according to an embodiment of the present invention.

Referring to FIG. 5, when air quality data measured by the air quality measurer 110 is input to the indoor air diagnotor 130, it is determined whether an ECO2 value is a fifth reference value (X₁) or more, a TVOC value is a sixth reference value (Y₁) or more, a PM10 value is a seventh reference value (Z₁) or more, a PM2.5 value is an eighth reference value (W₁) or more the second set number of times within the third set time (S501). In addition, in the case of the second set number of times within the third set time, it is detected as an abnormal situation (S502).

If it is not detected as an abnormal situation, air quality data goes through a smoking detection model (S503). As a result, when it is detected as smoking (S504), a coefficient is increased by 1 (S505), and when the coefficient becomes 3 or more (S506), the air quality data is entered into a smoking type classification model (S507). Accordingly, the smoking type is classified into a tobacco cigarette or an electronic cigarette (S508).

When smoking is not detected in S505, air quality data is used to determine whether an ECO2 value is the first reference value (X₂) or more and a TVOC value is the second reference value (Y₂) or more, or whether a PM10 value is a third reference value (Z₂) or more and a PM2.5 value is a fourth reference value (W₂) or more (S509). In addition, in the case of the first to fourth reference value or more, it is determined that the air quality is poor (S510), and if not, the air quality diagnosis process ends.

Referring to FIG. 1 again, the notification signal output 140 generates and outputs a notification signal according to the air diagnosis result of the indoor air diagnotor 130. For example, the notification signal output 140 may inform that there is a smoker by making a warning sound through a speaker, or may inform that there is a smoker by displaying it through a sign. Alternatively, the notification signal output 140 may transmit a short message or push message notifying that there is a smoker to a manager or the user's mobile 200 through wireless communication. Accordingly, a nearby administrator or user can quickly and accurately confirm that there is a smoker.

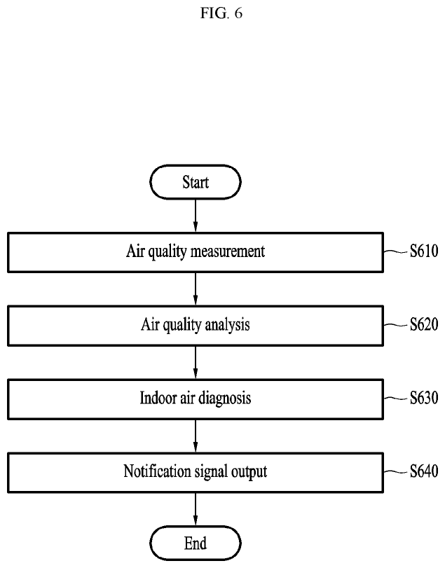

FIG. 6 is a flowchart for explaining a method of diagnosing indoor air quality according to an embodiment of the present invention. Referring to FIG. 6, when the method of diagnosing indoor air quality according to an embodiment of the present invention starts, indoor air quality in a limited space is first measured to obtain air quality data (S610). In addition, based on the machine learning result of the air quality data according to smoking or not, the obtained air quality data is analyzed (S620).

Next, one or more of indoor smoking and smoking types are diagnosed according to the air quality data analysis result (S630).

Next, a notification signal is generated and output according to the air diagnosis result of the indoor air diagnosis step (S640).

The method of diagnosing indoor air quality according to an embodiment of the present invention may be implemented by respective components of the system for diagnosis and management of indoor air quality described above, and since the method of diagnosing indoor air quality according to an embodiment of the present invention performs indoor air quality diagnosis in a similar manner to the system for diagnosis and management of indoor air quality described above, a specific description of the method of diagnosing indoor air quality according to an embodiment of the present invention is omitted to prevent duplication of explanation.

The method of diagnosing indoor air quality according to an embodiment of the present invention may be stored in a recording medium, such as CD-ROM, RAM, ROM, a floppy disk, a hard disk, a magneto-optical disk, a Secure Digital (SD) card, a micro SD card, or a Universal Serial Bus (USB) memory, implemented as a program and is readable by a computer.

The method of diagnosing indoor air quality according to an embodiment of the present invention may be implemented in the form of a web-based program or in the form of an application installed in a mobile terminal. In addition, a program in which the method of diagnosing indoor air quality according to an embodiment of the present invention is implemented may be a form installed in the system for diagnosis and management of indoor air quality according to an embodiment of the present invention.

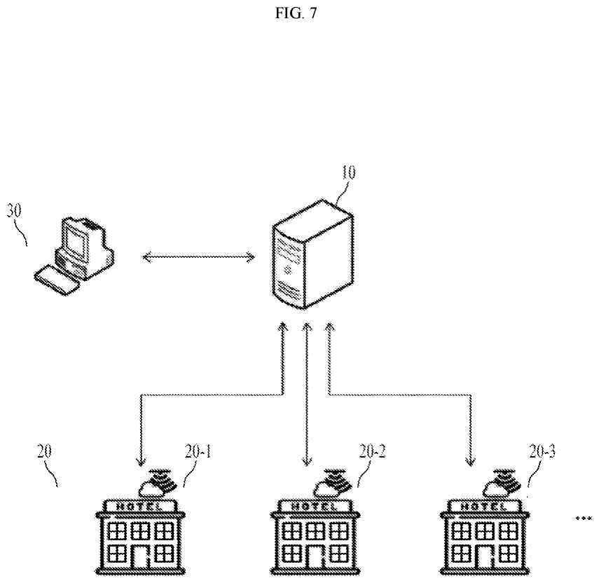

FIG. 7 is a reference diagram for explaining a system (hereinafter referred to as “management system”) for diagnosis and management of indoor air quality according to another embodiment of the present invention.

Referring to FIG. 7, the management system is a system applied to general accommodations and may include a service-providing device 10, an air quality-measuring device 20 that can be installed in each room of accommodation, and a manager terminal 30 for delivering accommodation-related information to a user (or a manager). The service-providing device 10 may be connected to the air quality-measuring device 20 and the manager terminal 30 through a network.

The service-providing device 10 corresponds to a computing device managed by a service provider who provides an accommodation management service using air quality analysis, and may be implemented as a server. Here, the service-providing device 10 may be connected to the air quality-measuring device 20 and the manager terminal 30 through a network, may analyze air quality data of accommodation collected from the connected air quality-measuring devices 20-1, 20-2, 20-3, and the like, and may transmit the analysis result and the customer type information according to the analysis result to the manager terminal 30, thereby providing an accommodation management service capable of integrating control over each accommodation room to be managed by a manager. Here, the air quality-measuring devices 20-1, 20-2, 20-3, and the like may be respectively installed in rooms 101, 102 and 103 of the accommodation. In addition, the manager terminal 30 is a computing device carried, managed or manipulated by an accommodation manager. The manager terminal 30 according to the present invention is a computing device that includes a display device and is used as a means to provide air quality control services to a manager, and may correspond to an electronic device, for example, such as smart phones, a tablet PC, and a desktop PC. However, these examples are not intended to limit the scope of rights of the present invention, and a computing device capable of providing visual information to a manager through a display device should be interpreted as the manager terminal 30 according to the present invention.

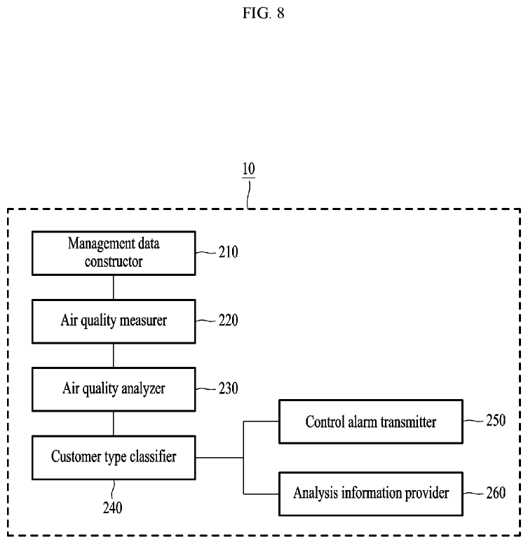

FIG. 8 is a block diagram illustrating the composition of a service-providing device 10 according to the present invention.

Referring to FIG. 2, the service-providing device 10 according to the present invention may include a management data constructor 210, an air quality measurer 220, an air quality analyzer 230 and a customer type classifier 240. In addition, the service-providing device 10 according to the present invention may further include a control alarm transmitter 250 and an analysis information provider 260 according to an embodiment.

The management data constructor 210 may construct management data for accommodation. In an embodiment, the management data constructor 210 may construct management data based on one or more of a customer's accommodation use history and smoking history. Here, Information included in management data to be constructed may be used in a process of classifying customer types according to an embodiment of the present invention.

The air quality measurer 220 may obtain air quality data for each room of the accommodation. In an embodiment, the air quality measurer 220 may collect air quality data from the air quality-measuring device 20 installed in each room of accommodation to measure air quality. Here, data collected from the air quality-measuring device 20 may include at least one of a fine dust concentration, a carbon dioxide concentration, and a volatile organic compound concentration. In addition, the air quality-measuring device 20 may include an air quality sensor (AQS). The air quality sensor is a sensor that measures the level of harmful substances such as ECO2, TVOC, PM10, and PM2.5 contained in the air, and air quality data collected from the air quality-measuring device 20 may be used for machine learning in the air quality analyzer 230 or used to analyze air quality data.

The air quality analyzer 230 may analyze the obtained air quality data based on machine learning results of the air quality data.

In an embodiment, the air quality analyzer 230 may diagnose an abnormal situation for each room of accommodation. For example, when an ECO2 value measured by the air quality-measuring device 20 is a first reference value or more and a TVOC value measured thereby is a second reference value or more or when a PM10 value is a third reference value or more and a PM2.5 value is a fourth reference value or more, the air quality of accommodation may be diagnosed as abnormal. That is, the air quality analyzer 230 may diagnose that air quality is abnormal rather than simply caused by smoking when a measurement value measured by the air quality-measuring device 20 exceeds a predetermined threshold value. As a specific example, the first reference value may be 1000 μg/m³, the second reference value may be 1300 μg/m³, the third reference value may be 80 μg/m³, and the fourth reference value may be 35 μg/m³. 80 μg/m³ as an example of the third reference value of PM10 and 35 μg/m³ as an example of the fourth reference value of PM2.5 are values selected according to the air quality standards specified by the Korea Meteorological Administration. On the other hand, in the case of ECO2 and TVOC, the ratings are not separately determined according to the Korea Meteorological Administration or international standards, so examples of the first reference value of ECO2 and the second reference value of TVOC were determined based on values calculated by averaging the observed values of TVOC and ECO2 when the observed air quality values PM10 and PM2.5 collected through experiments are at abnormal air quality state levels. These first to fourth reference values are not limited to the values given as examples, and the first to fourth reference values may be changed and set as needed.

The air quality analyzer 230 may diagnose the air quality of accommodation as being in a dangerous state when the ECO2 value measured by the air quality-measuring device 20 is a fifth reference value or more, the TVOC value is a sixth reference value or more, the PM10 value is a seventh reference value or more, and the PM2.5 value is an eighth reference value or more the first set number of times within the first set time. Here, the dangerous state may refer to a situation in which the concentration of harmful substances indoors becomes very high due to a fire or the like. As a specific example, the fifth reference value may be 3500 μg/m³, the sixth reference value may be 4000 μg/m³, the seventh reference value may be 1700 μg/m³, and the eighth reference value may be 1700 μg/m³. The examples of the fifth to eighth reference values were determined as examples of experimental measurement values when a carbon dioxide alarm is actually dangerous enough to sound. These fifth to eighth reference values are not limited to the values given as examples, and the fifth to eighth reference values may be changed and set as needed. Here, the fifth reference value is greater than the first reference value, the sixth reference value is greater than the second reference value, the seventh reference value is greater than the third reference value, and the eighth reference value is greater than the fourth reference value Here, The first set time or the first set number of times may be set to an appropriate value to diagnose a dangerous state, and may be changed and set as needed. For example, the first set time may be 2 minutes, and the first set number of times may be 3 times.

In an embodiment, the air quality analyzer 230 may include a classification model generator that generates a model for classifying types of air quality anomalies based on machine learning results for the obtained air quality data. The classification model generator may use one or more learning models selected from learning models including decision tree, random forest, Extreme Gradient Boosting (XGBOOST) and Support Vector Machine (SVM) to create a smoking detection model or a smoking type classification model.

In an embodiment, the classification model generator may create a smoking detection model that can detect smoking in an accommodation room and a smoking type classification model that can classify the type of smoking in the accommodation room. Specifically, the classification model generator may create a smoking detection model that can detect indoor smoking by learning smoking data, which is air quality data when smoking occurs indoors, and non-smoking data, which is air quality data when smoking does not occur indoors, or a smoking type classification model that can classify smoking types by learning tobacco data, which is air quality data when a tobacco cigarette smoking occurs indoors, and cigarette data which is air quality data when an electronic cigarette smoking occurs.

In an embodiment, the classification model generator may select an optimal parameter in one or more manners of grid search and cross validation in parameters (hyperparameters) applied to the learning model.

In an embodiment, the air quality analyzer 230 may determine that a customer using accommodation is smoking when smoking is detected the second set number of times within the second set time by the smoking detection model. For example, the air quality analyzer 230 may detect smoking by the smoking detection model. When 3 or more smoking is detected within 2 minutes, it may be determined that an accommodation user smokes. Here, the second set time or the second set number of times may be set to an appropriate value to determine whether the customer smokes, and may be changed and set as needed.

In an embodiment, when the air quality analyzer 230 determines that a customer using accommodation smokes, smoking may not be detected for the third set time from the time of the first smoking detection after detecting smoking the second set number of times. For example, when smoking is detected 3 times or more within 2 minutes and the customer is determined to be smoking, smoking may not be detected for 6 minutes from the time of the first smoking detection. The reason why smoking is not detected for a certain period of time is to prevent duplicate smoking detection for a single smoking act. In the example, the third set time is set to 6 minutes, which is a time set considering an average smoking time of 4 minutes and a normal air quality recovery time of 2 minutes. However, the third set time is not limited thereto, may be set to an appropriate value, or may be changed and set as needed.

In an embodiment, the air quality analyzer 230 diagnoses a final smoking type based on a smoking type classified through the smoking type classification model. Here, a smoking type showing a higher number of a tobacco cigarette classification number and an electronic cigarette classification number may be diagnosed as the final smoking type.

The customer type classifier 240 may classify the type of a customer using accommodation based on the analysis result of management data and air quality data. Here, the type of customer to be classified can be used to build management data, and when delivered to a manager who manages accommodation, the manager may use this to provide customized services according to each customer type.

In an embodiment, the customer type classifier 240 may classify customers into any one of a smoking customer group, a non-smoking customer group, and other customer groups. Here, the smoking customer group includes customers who smoke in accommodation, the non-smoking customer group includes customers who normally use accommodation or customers whose rooms are diagnosed with normal air quality, customers whose rooms are diagnosed with abnormal air quality, and customers diagnosed with air quality risks due to fire or abnormal behavior in accommodation, and other customer groups include customers in the case where there is no change in a value measured by an air quality sensor of the air quality-measuring device 20 installed in a room and in the case where the measured value is not transmitted to the service-providing device 10. Classification of each customer group may be done by basically reflecting the analysis result of the air quality analyzer 230. Here, for the customer type classification of the customer type classifier 240, logistic regression or cluster analysis may be used, which will be described in detail below.

In an embodiment, the customer type classifier 240 may further classify customers corresponding to the smoking customer group into a first smoking customer or a second smoking customer. Here, the first smoking customer may include a customer for whom a relatively small amount of smoking is detected compared to the second smoking customer, and a customer who has smoked more frequently through electronic cigarettes than tobacco cigarettes, and may refer to a customer who has an influence below the standard set in an accommodation room. In addition, the second smoking customer is a customer for whom a greater amount of smoking than the first smoking customer is detected, for example, a customer who has been detected several times of smoking and has an influence greater than or equal to a standard set in accommodation.

In an embodiment, the customer type classifier 240 may further classify customers belonging to the non-smoking customer group into normal customers, cautionary customers, and risky customers. Here, the normal customer may mean a customer who has been diagnosed with normal air quality in a room and normally uses accommodation according to a set rule, and the cautionary customer may mean a customer diagnosed as having abnormal air quality in a room. In addition, the risky customer may refer to a customer experiencing an abnormal situation, such as a customer diagnosed with an air quality risk due to a fire or abnormal behavior in accommodation.

In an embodiment, the customer type classifier 240 may classify customers corresponding to each customer group through logistic regression or cluster analysis. As a specific example, the customer type classifier 240 may perform logistic regression or cluster analysis by applying independent variables extracted from management data. Here, the independent variables extracted from the management data may include the number of times a customer has used accommodation in the past, use periods of accommodations, the locations of customers in accommodation, the type of room a customer uses, a customer's smoking history (including smoking frequency), a current customer's smoking frequency, the installation date of the quality-measuring device 20, the software information of the air quality-measuring device 20, and the like.

In addition, The customer type classifier 240 may perform logistic regression or cluster analysis by setting a criterion for classifying customers as a dependent variable. According to an embodiment of the present invention, the criterion for classifying customers may be the standard deviation.

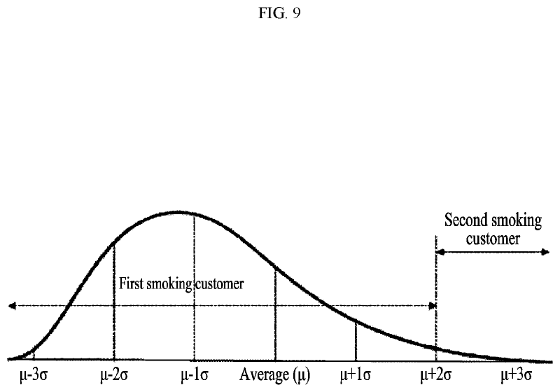

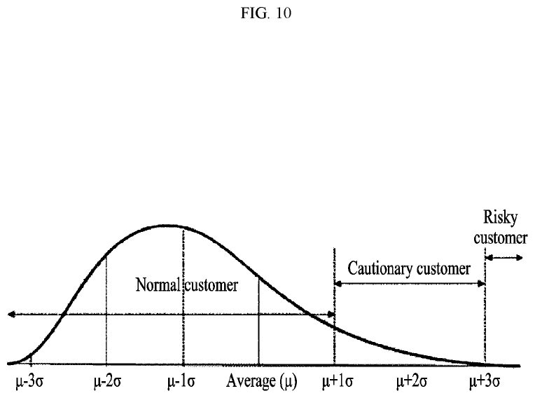

In this regard, FIGS. 9 and 10 are reference diagrams for explaining a process of classifying customer types using accommodation according to another embodiment of the present invention.

Referring to FIGS. 9 and 10, in an embodiment, the customer type classifier 240 performs the logistic regression or the cluster analysis to subclass a customer, who corresponds to an interval with a standard deviation twice or more than an average, among customers belonging to the smoking customer group into the second smoking customer, and performs the logistic regression or the cluster analysis to subclass a customer, who falls within an interval excluding intervals that are twice the standard deviation or more than an average, among customers belonging to the smoking customer group into the first smoking customer. In addition, the customer type classifier 240 performs the logistic regression or the cluster analysis to subclass a customer, who falls into the range of 3 times a standard deviation or more than an average, among customers belonging to the non-smoking group, into the risky customer, and performs the logistic regression or the cluster analysis to subclass a customer, who falls within an interval excluding an interval that is 1 times the standard deviation or more than an average, among customers belonging to the non-smoking customer group into the normal customer.

The control alarm transmitter 250 may deliver a control alarm for each room of accommodation to the customer based on the type of customer to be classified.

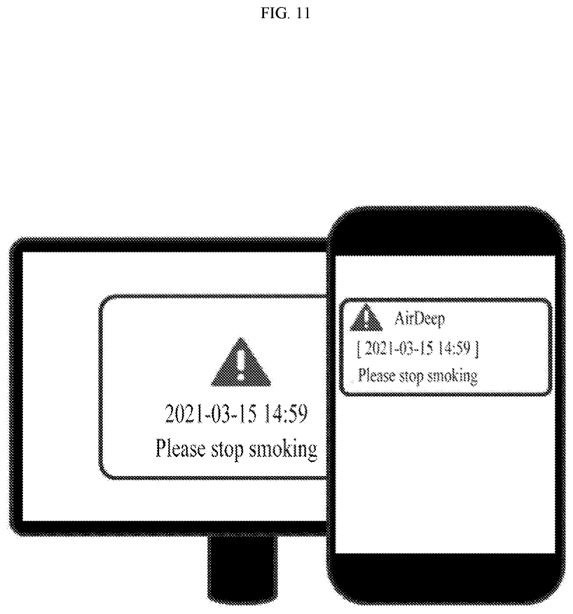

In this regard, FIG. 11 is a reference diagram illustrating a control alarm delivered to a customer using accommodation according to another embodiment of the present invention.

Referring to FIG. 11, the control alarm transmitter 250 may deliver control alarms to customers through display devices (e.g., devices capable of outputting control alarms such as TVs and PCs) provided in each room of accommodation, and also deliver control alarms through customer's terminals. The control alarm may suppress fraudulent behavior such as smoking in customer's rooms, and even in the event of a dangerous situation such as fire or increased concentration of harmful substances, the control alarm informs the customer, so that the customer in the room can respond quickly.

The analysis information provider 260 may provide reporting-type analysis information to the accommodation manager based on the classified customer type.

In an embodiment, the analysis information provider 260 may transmit failure reporting-type analysis information related to problems occurring in customer rooms, in which customer groups are classified, to a manager. For example, in the case of a fire in a room, reporting analysis information on the fire outbreak is sent to the manager via e-mail or the manager terminal 30, so that the manager can identify problems in the accommodation in real-time and take action.

In an embodiment, the analysis information provider 260 may provide reporting-type analysis information including a customer's smoking history to a manager. Through this, the manager can monitor each customer's smoking history and design a customized service to be provided to each customer. For example, the manager may apply a method of granting a penalty for using accommodation to customers with an excessive smoking history, or may design an accommodation provision service of granting benefits to customers who use accommodation a certain number of times or more normally.

Although specific embodiments of the present invention have been shown and described above, it will be obvious to those skilled in the art that the technical idea of the present invention is not limited to the accompanying drawings and the above description, and various forms of modification are possible within the scope of not departing from the spirit of the present invention. Such modifications will be deemed to belong to the claims of the present invention in a scope where they do not violate the idea of the present invention.

1. A system for diagnosis and management of indoor air quality, comprising:
an air quality measurer configured to obtain air quality data by measuring indoor air quality in a limited space;
an air quality analyzer configured to analyze the obtained air quality data based on a machine learning result of the air quality data according to smoking or not; and
an indoor air diagnotor configured to diagnose one or more of indoor smoking or not and smoking types based on the air quality data analysis result,
wherein the air quality analyzer comprises a detection model generator that creates a smoking detection model capable of detecting indoor smoking by learning smoking data, which is air quality data when smoking is performed indoors, and non-smoking data, which is air quality data when smoking is not performed indoors and that creates a smoking type classification capable of classifying smoking types by learning tobacco data, which is air quality data when smoking is performed with a tobacco cigarette indoors, and cigarette data which is air quality data when smoking is performed with an electronic cigarette model.

2. The system according to claim 1, wherein the detection model generator creates the smoking detection model or the smoking type classification model using one or more models selected from supervised learning models comprising decision tree, random forest, Extreme Gradient Boosting (XGBOOST) and Support Vector Machine (SVM).

3. The system according to claim 1, wherein the indoor air diagnotor determines that there is a smoker indoors when smoking is detected a first set number of times within a first set time by the smoking detection model,
classifies a smoking type by the smoking type classification model, sums the number of times classified as a tobacco cigarette and the number of times classified as an electronic cigarette, and diagnoses a smoking type based on the number of times more than half of the number of times classified as a tobacco cigarette and the number of times classified as an electronic cigarette, and
when determining that there is a smoker indoors, does not detect smoking for a second set time from a first smoking detection point after the first set number of times of smoking detection.

4. The system according to claim 1, wherein the air quality measurer is an air quality sensor (AQS).

5. The system according to claim 1, wherein the detection model generator selects an optimal parameter by one or more of grid search and cross validation in parameters (hyperparameters) applied to the supervised learning mode.

6. The system according to claim 1, wherein the indoor air diagnotor determines that air quality is poor when an ECO2 value measured by the air quality measurer is a first reference value and a TVOC value measured thereby is a second reference value or more or when a PM10 value is a third reference value or more and a PM2.5 value is a fourth reference value or more.

7. The system according to claim 6, wherein the first reference value is 1000 μg/m³, the second reference value is 1300 μg/m³, the third reference value is 80 μg/m³, and the fourth reference value is 35 μg/m³.

8. The system according to claim 1, wherein the indoor air diagnotor determine as an abnormal situation when an ECO2 value measured by the air quality measurer is confirmed as a fifth reference value or more, a TVOC value is confirmed as a sixth reference value or more, a PM10 value is confirmed as a seventh reference value or more, and a PM2.5 value is confirmed as an eighth reference value or more a second set number of times within a third set time.

9. The system according to claim 8, wherein the fifth reference value is 3500 μg/m³, the sixth reference value is 4000 μg/m³, the seventh reference value is 1700 μg/m³, and the eighth reference value is 1700 μg/m³.

10. The system according to claim 1, further comprising a notification signal output configured to generate and output a notification signal according to an air diagnosis result of the indoor air diagnotor.

11. A system for diagnosis and management of indoor air quality, comprising:
an air quality measurer configured to obtain air quality data for each room in accommodation;
an air quality analyzer configured to create a smoking detection model and a smoking type classification model and analyze the obtained air quality data based on the machine learning results of the air quality data;
a management data constructor configured to construct management data for the accommodation; and
a customer type classifier configured to classify types of customers using the accommodation based on the analysis results of the management data and the air quality data,
wherein the customer type classifier classifies the customer into any one of a smoking customer group, a non-smoking customer group and other customer groups, subclasses a customer corresponding to the smoking customer group into a first smoking customer or a second smoking customer, and subclasses customers belonging to each customer group through logistic regression or cluster analysis.

12. The system according to claim 11, wherein the management data constructor constructs the management data based on one or more of an accommodation use history of the customer and a smoking history of the customer.

13. The system according to claim 11, wherein the customer type classifier subclasses customers corresponding to the non-smoking customer group into a normal customer, a cautionary customer, or a risky customer.

14. The system according to claim 11, wherein the customer type classifier performs the logistic regression or cluster analysis by applying independent variables extracted from the management data.

15. The system according to claim 11, wherein the customer type classifier sets a criteria for subclassing the customers as a dependent variable and performs the logistic regression or cluster analysis.

16. The system according to claim 11, wherein the customer type classifier performs the logistic regression or the cluster analysis to subclass a customer, who corresponds to an interval with a standard deviation twice or more than an average, among customers belonging to the smoking customer group into the second smoking customer, and
performs the logistic regression or the cluster analysis to subclass a customer, who falls within an interval excluding intervals that are twice the standard deviation or more than an average, among customers belonging to the smoking customer group into the first smoking customer.

17. The system according to claim 11, wherein the customer type classifier performs the logistic regression or the cluster analysis to subclass a customer, who falls into the range of 3 times a standard deviation or more than an average, among customers belonging to the non-smoking group, into the risky customer, and
performs the logistic regression or the cluster analysis to subclass a customer, who falls within an interval excluding an interval that is 1 times the standard deviation or more than an average, among customers belonging to the non-smoking customer group into the normal customer.

18. The system according to claim 11, further comprising:
a control alarm transmitter configured to deliver a control alarm for each room of the accommodation to the customer based on the type of customer to be classified; and
an analysis information provider configured to provide reporting-type analysis information to the accommodation manager based on the classified customer type.
